(19) United States
(12) Patent Application Publication
(10) Pub. No.: US 2012/0289971 A1
(43) Pub. Date: Nov. 15, 2012

(54) MULTIPLE LUMEN RETRIEVAL DEVICE AND METHOD OF USING

(75) Inventors: James D. Segermark, Gem Lake, MN (US); David R. Hewitt, Eden Prairie, MN (US)

(73) Assignee: MERIT MEDICAL SYSTEMS, INC., South Jordan, UT (US)

(21) Appl. No.: 13/105,653

(22) Filed: May 11, 2011

(51) Int. Cl. A61B 17/00

(52) U.S. Cl. 606/108

(57) ABSTRACT

A retrieval device with multiple internal lumens and one or more balloons is disclosed. The balloons may be utilized to stabilize the retrieval device during therapy and to prevent migration of the device, or material within a body lumen, during therapy.

### TECHNICAL FIELD

The present disclosure relates generally to devices used to retrieve, cut, or otherwise manipulate items or structures which may be located in anatomically remote locations, such as items located in body lumens. More specifically, the present disclosure relates to retrieval devices, such as snares, which may be configured with multiple internal lumens and one or more balloons.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a side view of a retrieval device catheter having two internal lumens and one balloon.

FIG. 1A is a cross sectional view of the retrieval device catheter of FIG. 1 taken through lines X-X.

FIG. 1B is an alternate cross sectional view of the retrieval device catheter of FIG. 1 taken through lines X-X.

FIG. 1C is an alternate cross sectional view of the retrieval device catheter of FIG. 1 taken through lines X-X.

FIG. 2A is a side view of a retrieval device catheter having two internal lumens and one balloon.

FIG. 2B is a side view of a retrieval device having two lumens and one balloon.

FIG. 3 is a side view of a retrieval device having three lumens and one balloon.

FIG. 3A is a cross sectional view of the retrieval device of FIG. 3 taken through lines Y-Y.

FIG. 3B is an alternate cross sectional view of the retrieval device of FIG. 3 taken through lines Y-Y.

FIG. 3C is an alternate cross sectional view of the retrieval device of FIG. 3 taken through lines Y-Y.

FIG. 3D is an alternate cross sectional view of the retrieval device of FIG. 3 taken through lines Y-Y.

FIG. 4 is a side view of a retrieval device having three lumens and two balloons.

FIG. 5 is a side view of a retrieval device having four lumens and two balloons.

FIG. 5A is a cross sectional view of the retrieval device of FIG. 5 taken through lines Z-Z.

FIG. 5B is an alternate cross sectional view of the retrieval device of FIG. 5 taken through lines Z-Z.

FIG. 5C is an alternate cross sectional view of the retrieval device of FIG. 5 taken through lines Z-Z.

FIG. 6A is a side view of a retrieval device catheter having two balloons.

FIG. 6B is a side view of the retrieval device catheter of FIG. 6A in an alternate configuration.

FIG. 6C is a side view of the retrieval device catheter of FIG. 6A in another alternate configuration.

FIG. 7 is a side view of a retrieval device in use within a body lumen.

### DETAILED DESCRIPTION

A retrieval device may be configured with multiple internal lumens and one or more balloons. Balloons may be utilized to stabilize a retrieval device during therapy or to prevent migration of fragments or other materials within a body lumen. One or more balloons may establish a stable platform from which a practitioner may deploy a cutting or capturing device, such as a snare. Precise positioning may enable a practitioner to more quickly and efficiently perform the needed therapy. Further, precise positioning may lessen trauma at the therapy site, minimizing injury from unwanted contact between portions of the retrieval device and portions of the body lumen. For example, precise positioning and stable movement of a snare loop may reduce the possibility that the snare loop will rotate (or “whip”) during therapy, which rotation can, for example, damage the inner lining of blood vessels.

Multiple lumens may add functionality to a retrieval device. Each lumen may be configured to accommodate a device or perform a function useful for therapy. For instance, a retrieval device may include a lumen configured to accommodate a snare, a second lumen configured to accommodate a guidewire, and a third lumen configured to function in connection with a balloon. The lumens mentioned in this paragraph are exemplary only and a device may omit any of these lumens, include multiple lumens which are all configured to operate in connection with the same type of tool or therapy, or include other lumens configured to operate in connection with tools or therapies not listed in the example described above (e.g., a multiple lumen retrieval device may have a snare lumen, two balloon lumens, no guidewire lumen, and may or may not include other lumens adapted for other purposes known in the art).

It will be readily understood that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in the drawings, the drawings are not necessarily drawn to scale unless specifically indicated.

“Retrieval device” is used broadly herein and is not limited simply to devices which may surround or secure material within a body lumen (such as a snare loop). As used herein, a retrieval device is a medical instrument configured to cut, capture, surround, reposition, hook, snare, or otherwise interact with materials within the human body, including materials in body lumens. Furthermore, the disclosure provided herein is relevant to any type of minimally invasive therapy performed on the human body. Though examples below may refer to use of the device with body lumens, the disclosure is also relevant for devices configured to be disposed within the human body, though not necessarily within lumens. Likewise, though particular examples may refer to particular body lumens, such as those of the central venous system, the disclosure is relevant to any body lumen, such as the biliary duct, the esophagus, the trachea, and so on.

The phrases “connected to,” “coupled to,” and “in communication with” refer to any form of interaction between two or more entities, including mechanical, electrical, magnetic, electromagnetic, fluid, and thermal interaction. Two components may be coupled to each other, even though they are not in direct contact with each other. For example, two components may be coupled to each other through an intermediate component. Additionally, the term “abuts” refers to items that are in direct physical contact with each other, although the items may not necessarily be attached together.

The directional terms “distal” and “proximal” are given their ordinary meaning in the art. That is, the distal end of a medical device means the end of the device furthest from the practitioner during use. The proximal end refers to the opposite end, or the end nearest the practitioner during use.

“Delivery conduit,” as used herein, refers to an artificial channel capable of establishing communication between a remote location and an external environment. For example, in certain embodiments described herein, the delivery conduit comprises the outer sheath of a snare device, which in some embodiments comprises a catheter.

A used herein, “fragment” refers to the target of retrieval device-type therapy. For example, a fragment may be a foreign object disposed within a body lumen or an anatomical structure within the body which requires ligation or removal.

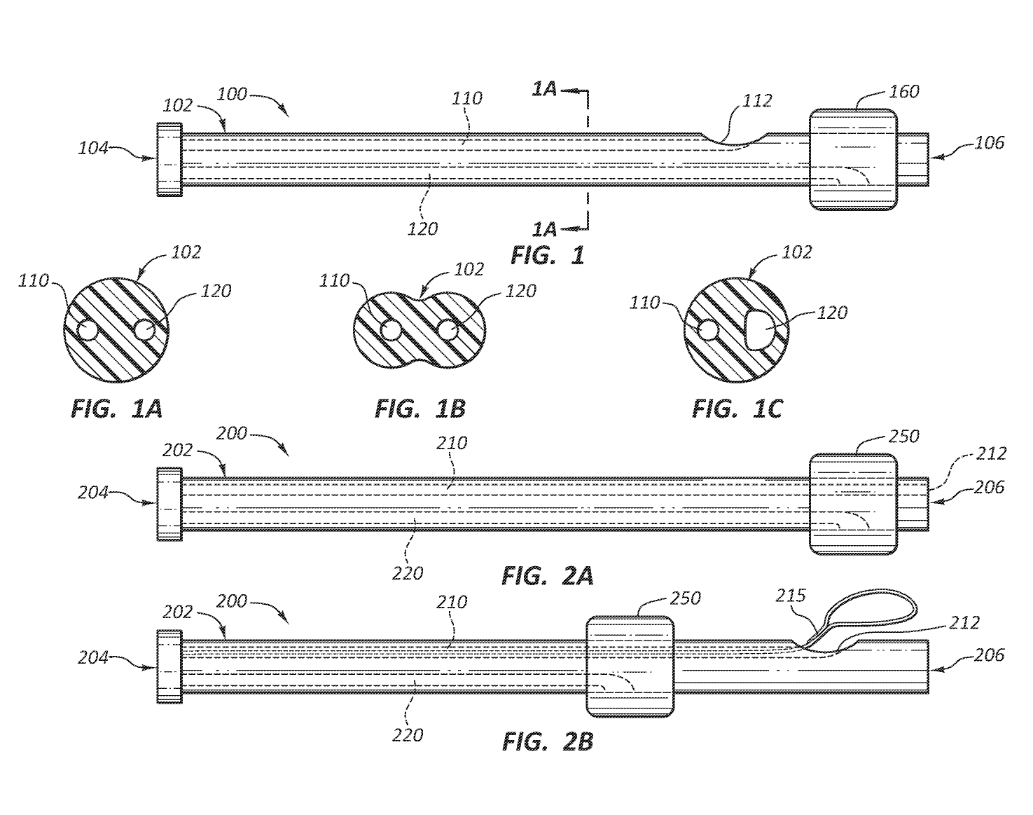

FIG. 1 is a side view of a retrieval device 100 including an outer sheath or catheter 102 having proximal 104 and distal ends 106. In the illustrated embodiment, the catheter 102 has a first lumen 110, which may be a device lumen, configured to receive a medical device capable of cutting or capturing fragments disposed within the body of a patent. The device lumen 110 may be configured to receive any medical device or tool capable of cutting or capturing material.

In certain embodiments, the catheter 102 may also be configured with a connector at its proximal end 104 configured to couple the catheter 102 to another device. This connector may be any type of connector known in the art, for example, a luer connector.

The device lumen 110 may have an opening 112 adjacent the distal end 106 of the catheter 102. As illustrated, the distal opening 112 of the device lumen 110 may be located in a sidewall of the catheter 102. In other embodiments, the distal opening 112 of the device lumen 110 may be located in the distal end 106 of the catheter 102. The device lumen 110 may also have a proximal opening (not shown) to allow a practitioner to access and manipulate the proximal end of an elongate medical device disposed within the device lumen 110.

The retrieval device of FIG. 1 also includes a second lumen 120, which may be in fluid communication with a first balloon 160. The second lumen 120 may also be referred to as the first balloon lumen 120, which may allow a practitioner to inflate and/or deflate the first balloon 160. The first balloon lumen 120 may have a distal opening, which is in fluid communication with the first balloon 160 and a proximal opening at which a practitioner can introduce (or reduce) fluid pressure in order to inflate (or deflate) the first balloon 160.

In certain embodiments, such as that illustrated in FIG. 1, the first balloon 160 may be located between the distal opening 112 of the device lumen 110 and the distal end 106 of the catheter 102. In other embodiments, the distal opening 112 of the device lumen 110 may be located between the first balloon 160 and the distal end 106 of the catheter 102.

The first balloon 160 may be configured to expand radially outward from the catheter 102. In some embodiments, the first balloon 160 will be configured to expand outwardly such that the first balloon 160 contacts the wall of a body lumen within which the retrieval device is disposed. Further, the first balloon 160 may be configured such that it has an unexpanded or undeployed state. In the undeployed state (not shown), the first balloon 160 may be disposed such that it abuts the outer surface of the catheter 102. In some embodiments, the first balloon 160 in the undeployed state will not substantially extend beyond the outside diameter of the catheter 102. In other words, the first balloon 160 may be configured such that, when undeployed, it does not significantly increase the overall cross section or profile of the catheter 102.

The first balloon 160 may be configured to stabilize the retrieval device 100 during therapy. For example, the retrieval device 100 may be disposed within a body lumen of a patient while the first balloon 160 is in an undeployed state. The first balloon 160 could then be deployed such that it expands radially outward until it contacts the walls of the surrounding body lumen. Contact between the first balloon 160 and the body lumen may stabilize the retrieval device 100 with respect to the body lumen. For instance, contact between the first balloon 160 and the body lumen may minimize or prevent rotation of the retrieval device 100 with respect to the body lumen. Contact between the first balloon 160 and the lumen wall may also tend to maintain the position of the catheter 102 in the center of the body lumen and maintain the position of the catheter 102 along the axis of the body lumen.

Stabilization of the catheter 102 with respect to the body lumen may tend to lessen unwanted trauma during therapy. Movement of the catheter 102, with respect to the body lumen during therapy, may result in unwanted contact between a cutting or capturing device (such as a device extending from the distal opening 112 of the device lumen 110) and the body lumen. Essentially, contact between the first balloon 160 and the body lumen may provide a stable platform from which to deploy a cutting or capturing medical device. The distance between the distal opening 112 of the device lumen 110 and the first balloon 160 may be configured to provide optimal stabilization while maintaining sufficient offset to prevent the first balloon 160 from obscuring the therapy site. The optimal distance may be different in different applications. Likewise, whether it is optimal to position the first balloon 160 proximally or distally to the distal opening 112 of the device lumen 110 may depend on the type of therapy and the location of the therapy site within the body.

The first balloon 160 may also be utilized to prevent migration of fragments of other material within the body lumen during therapy. For example, in a therapy being performed within the central venous system, the retrieval device 100 may be positioned such that blood flowing within the lumen flows from proximal end 104 of the catheter 102 toward the distal end 106. Further, the first balloon 160 may be coupled to the catheter 102 distal of the distal opening 112 of the device lumen 110. In this example, blood flowing past the distal opening 112 of the device lumen flows toward the first balloon 160. When the device is position as described by this example, fragments or other materials dislodged but not initially captured could be prevented from migrating along the body lumen, as such fragments would be trapped against the first balloon 160. The practitioner could subsequently capture or aspirate these fragments, if desired, before the first balloon 160 is deflated. (It will be appreciated that, depending on the access point, in some instances the first balloon 160 will be positioned distal of the distal opening 112, and in other instances, the first balloon 160 will be proximal of the distal opening 112 in order for the first balloon 160 to be located such that blood flowing past the distal opening 112 of the device lumen 110 flows toward the first balloon 160.)

Similarly, the first balloon 160 may be used to restrict flow through a body lumen, such as a lumen of the central venous system, during therapy. The first balloon 160 may slow or stop the flow of blood or other fluid through the body lumen, which may further stabilize the therapy site. In some embodiments, the first balloon 160 may be positioned such that blood flowing past the distal opening 112 of the device lumen 110 flows away from the first balloon 160. When so arranged, inflation of the first balloon 160 may restrict or stop blood from flowing past the distal opening 112 of the device lumen 110. In some instances a practitioner may inflate the first balloon 160, perform one or more portions of the therapy, deflate the first balloon 160 to allow flow (and pressure reduction around the balloon), then reinflate the first balloon 160 and continue therapy. These steps may be repeated multiple times.

FIGS. 1A, 1B, and 1C are exemplary cross sections of the retrieval device of FIG. 1, taken through line X-X. These views are exemplary only; it will be appreciated that any cross section known in the art may be utilized. For example, the catheter 102 may have a circular cross section (as in FIGS. 1A and 1C) or a “figure 8” type cross section (as in FIG. 1B). Similarly, the lumens 110, 120 may have circular, semicircular, or other cross sectional profiles. In some embodiments, the cross section of the first balloon lumen 120 may be semicircular; such a design may allow for increased flow to the first balloon 160 without substantially increasing the overall profile of the catheter 102.

FIGS. 2A and 2B illustrate two possible embodiments of a retrieval device (designated 200 and 200′, respectively), which can, in certain respects, resemble the retrieval device 100 described above. It will be appreciated that all the illustrated embodiments have analogous features. Accordingly, like features are designated with like reference numerals, with the leading digits incremented to “2.” Relevant disclosure set forth above regarding similarly identified features thus may not be repeated hereafter. Moreover, specific features of the retrieval device 200, 200′ may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the retrieval devices 200, 200′. Any suitable combination of the features, and variations of the same, described with respect to retrieval device 100, can be employed with retrieval device 200, 200′, and vice versa. This pattern of disclosure applies equally to further embodiments depicted in subsequent figures and described hereafter.

As shown in FIG. 2A, a retrieval device 200 may comprise an outer sheath or catheter 202 with two internal lumens 210, 220. The first lumen 210 may be a device lumen configured to accommodate a medical device. The device lumen may have a distal opening 212 located in the distal end 206 of the catheter 202. As illustrated in FIG. 2A, this opening 212 may be located distally of a first balloon 250.

FIG. 2B illustrates a retrieval device 200′ having two internal lumens 210′, 220′ and a first balloon 250′ coupled to the catheter 202′ proximally of the distal opening 212′ of the device lumen 210′. The disclosure made in connection with the first balloon 160 of FIG. 1 is applicable to the first balloon 250, 250′ of FIGS. 2A and 2B. Here and throughout this disclosure, balloons designated with the final two digits “50” are balloons positioned proximally of the distal opening of the device lumen, while balloons with the final two digits “60” are positioned distally of the distal opening of the device lumen. As described herein, however, the disclosure relative to a distally located balloon is applicable to a proximally located balloon, and vice versa.

The retrieval device of FIG. 2B further comprises a snare 215′ disposed within the device lumen 210′. The illustrated snare 215′ has an elongate portion disposed within the device lumen 210′ and a snare loop coupled to the distal end of the elongate portion. In other embodiments, the retrieval device 200′ may be designed to accommodate other capturing or cutting medical devices.

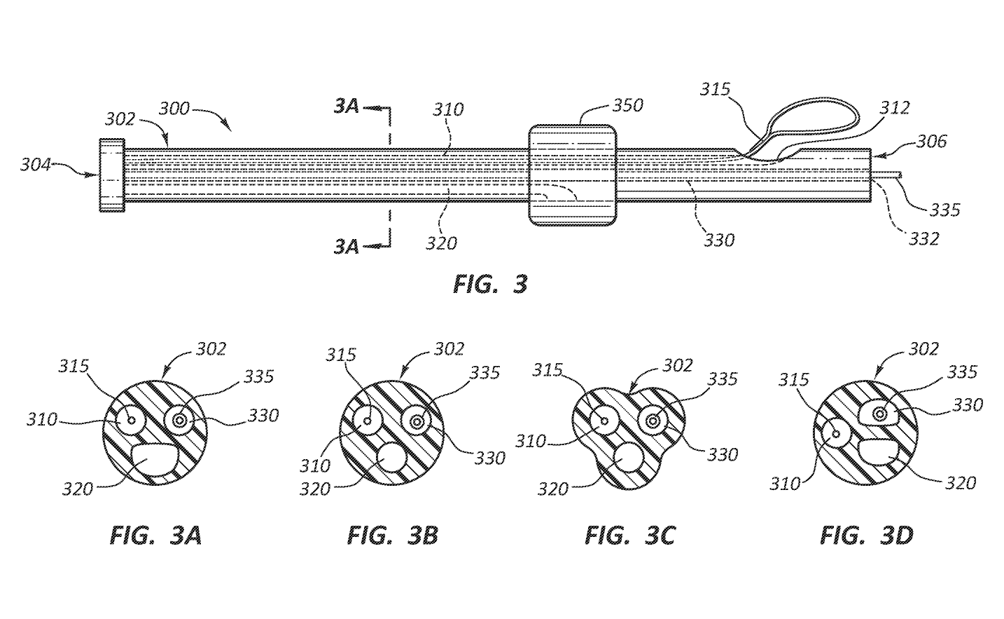

FIG. 3 is a side view of another embodiment of a retrieval device 300. Retrieval device 300 comprises an outer sheath or catheter 302 having proximal 304 and distal 306 ends. As illustrated in FIG. 3, retrieval device 300 may include three internal lumens: a first lumen 310, which may be a device lumen; a second lumen 320, which may be in fluid communication with a first balloon 350; and a third lumen 330, which may be a guidewire lumen. In the illustrated embodiment, a snare 315 is disposed within, and partially deployed from, the device lumen 310 and a guidewire 335 is disposed within the guidewire lumen 330.

A retrieval device may utilize a guidewire to aid in proper placement of the device within a body lumen. For example, a practitioner may first insert guidewire 335 into a body lumen of a patient. The practitioner may then slide the distal end of the catheter 302 over the proximal end of the guidewire 335, through a distal opening 332 in the guidewire lumen 330. The practitioner may then slide the catheter 302 along the guidewire 335 until the retrieval device 300 is at a desired location. The first balloon 350 may be in a deflated, or undeployed state, while the catheter 302 is being inserted into the body lumen. Further, the snare 315 may be completely disposed within the device lumen 310 while the catheter 302 is being inserted. In other instances, the practitioner may insert the catheter 302 without the snare 315, inserting the snare into the device lumen at the proximal end 304 of the catheter 302, after the catheter is in place within the body lumen. The practitioner may then inflate and deflate the first balloon 350 and deploy and retrieve the snare 315 as necessary to perform the therapy. The retrieval device may then be withdrawn from the body lumen.

Each lumen of the retrieval device 300 may be sized for its particular function. The device lumen 310 may be sized to accommodate any number of medical instruments. In some embodiments, the device lumen will be sized to accommodate a snare device 315; in such instances the diameter of the device lumen 310 may be from: about 0.025 inches to about 0.055 inches, including diameters from about 0.035 inches to about 0.045 inches; from about 0.055 inches to about 0.090 inches, including diameters from about 0.070 inches to about 0.085 inches; or from about 0.080 inches to about 0.110 inches, including diameters from about 0.085 inches to about 0.095 inches. The first balloon lumen 320 may likewise be sized to work in conjunction with balloons of various sizes, adapted to be used in various parts of the body. In some instances, the first balloon lumen may be from about 0.002 inches to about 0.009 inches in diameter. Further, in some embodiments, more than one lumen may be required to achieve desired balloon inflation and deflation times. Finally, the guidewire lumen 330 may be sized so as to accommodate a variety of guidewires configured to aid in accessing various parts of the body. In some instances, the guidewire lumen 330 will accommodate guidewires from about 0.009 inches to about 0.038 inches in diameter, which in some embodiments will correlate with guidewire lumen diameters from about 0.012 inches to about 0.044 inches.

FIGS. 3A-3D illustrate exemplary cross sectional profiles of retrieval device 300, taken through lines Y-Y. These views are exemplary only; it will be appreciated that any cross section known in the art may be utilized. As shown, the catheter 302 may have a circular cross section as in FIGS. 3A, 3B, and 3D; a clover shaped cross section as in FIG. 3C; or any other profile as known in the art. Further, each of the lumens may be circular in cross section, semi-circular, or any other cross sectional shape. For example, in FIG. 3A the device lumen 310 and the guidewire lumen 330 have circular cross sections while the first balloon lumen 320 has a semicircular cross section. As shown in FIGS. 3A-3D, the lumens could also be configured such that the device lumen 310 or the guidewire lumen 330 has a semicircular cross section and the balloon lumen 320 has a circular cross section. Any combination of these or other cross sectional shapes known in the art is within the scope of this disclosure.

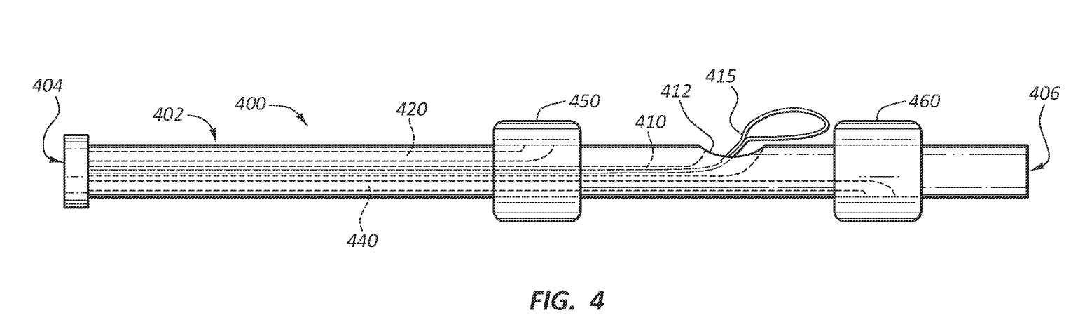

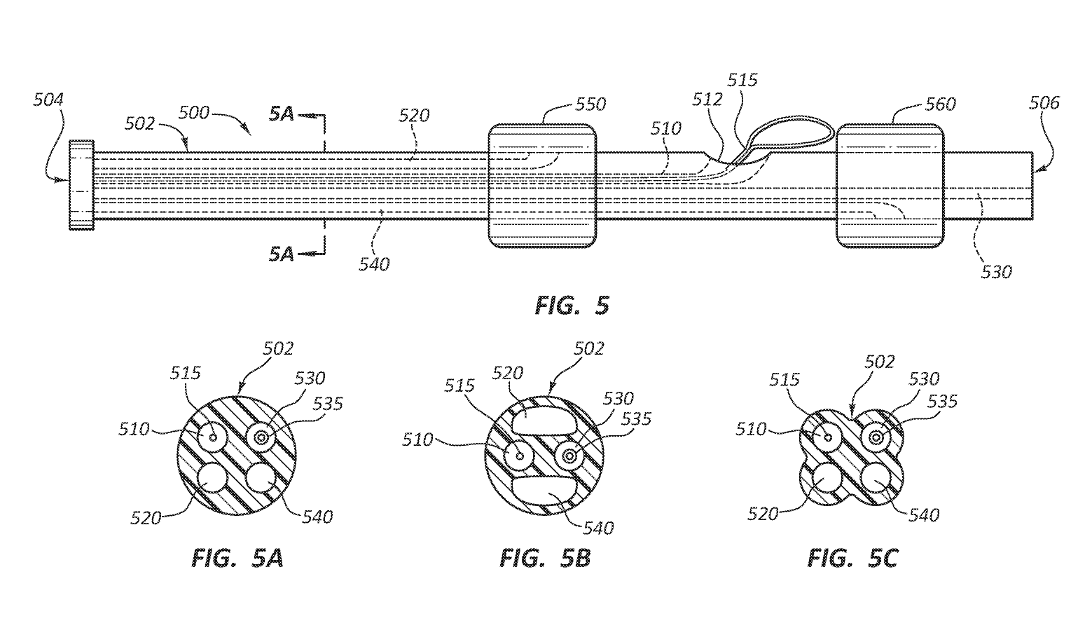

FIG. 4 is a side view of yet another embodiment of a retrieval device 400. In the illustrated embodiment, the retrieval device 400 includes a catheter 402 having proximal 404 and distal ends 406. A first lumen 410, which in some instances will be a device lumen, may be disposed within the catheter 402. Likewise, a second lumen 420, which may be a first balloon lumen, may be disposed within the catheter 402. The illustrated embodiment has three total lumens; in the illustrated embodiment the final lumen 440 is a second balloon lumen. The second balloon lumen 440 is designated as “440” to distinguish it from the guidewire lumen (designated 330 and 530) in other embodiments, and to associate it with the second balloon lumen of other embodiments (designated 540 in FIG. 5 and FIGS. 5A-5C).

The second balloon lumen 440 may be in fluid communication with a second balloon 460, which may be located distally of the distal opening 412 of the device lumen 410. It will be appreciated that, while the second balloon lumen 440 is illustrated as being in fluid communication with the distally located balloon 460, in other embodiments, it may instead be in fluid communication with the proximally located balloon 450. Likewise, the first balloon lumen 420 may be in fluid communication with the proximally located balloon 450 in some embodiments, and the distally located balloon 460 in other embodiments.

Two balloons, such as those illustrated in FIG. 4, may be used in conjunction with each other to stabilize the retrieval device at the therapy site. A practitioner may inflate one balloon and not the other, inflate both balloons, inflate both balloons then deflate only one, or any other combination of inflated/deflated states over the course of a single therapy. Though no exemplary cross sections are shown in connection with FIG. 4, it will be appreciated that the disclosure provided throughout, including the disclosure provided in connection with FIGS. 3A-3D, is applicable to the embodiment of FIG. 4.

FIG. 5 is a side view of yet another embodiment of a retrieval device 500. In the illustrated embodiment the retrieval device has an outer sheath or catheter 502 with a proximal end 504 and a distal end 506. Four lumens 510, 520, 530, 540 are positioned within the catheter 502. The first lumen 510 is a device lumen, and an elongate snare 515 is illustrated as disposed within, and partially deployed from, the device lumen 510. The second lumen 520 is a first balloon lumen in fluid communication with the proximal balloon 550. The third lumen 530 is a guidewire lumen and a guidewire 535 is illustrated as disposed within this lumen. Finally, the fourth lumen 540 is a second balloon lumen which is in fluid communication with the distally located balloon 560.

FIGS. 5A, 5B, and 5C are exemplary cross sectional views of the retrieval device 500 of FIG. 5 taken through lines Z-Z. As with the other exemplary cross sections disclosed above, the catheter 502 and each of the lumens 510, 520, 530, 540 can take on any of the shapes or profiles disclosed herein, as well as other configurations and shapes as known in the art.

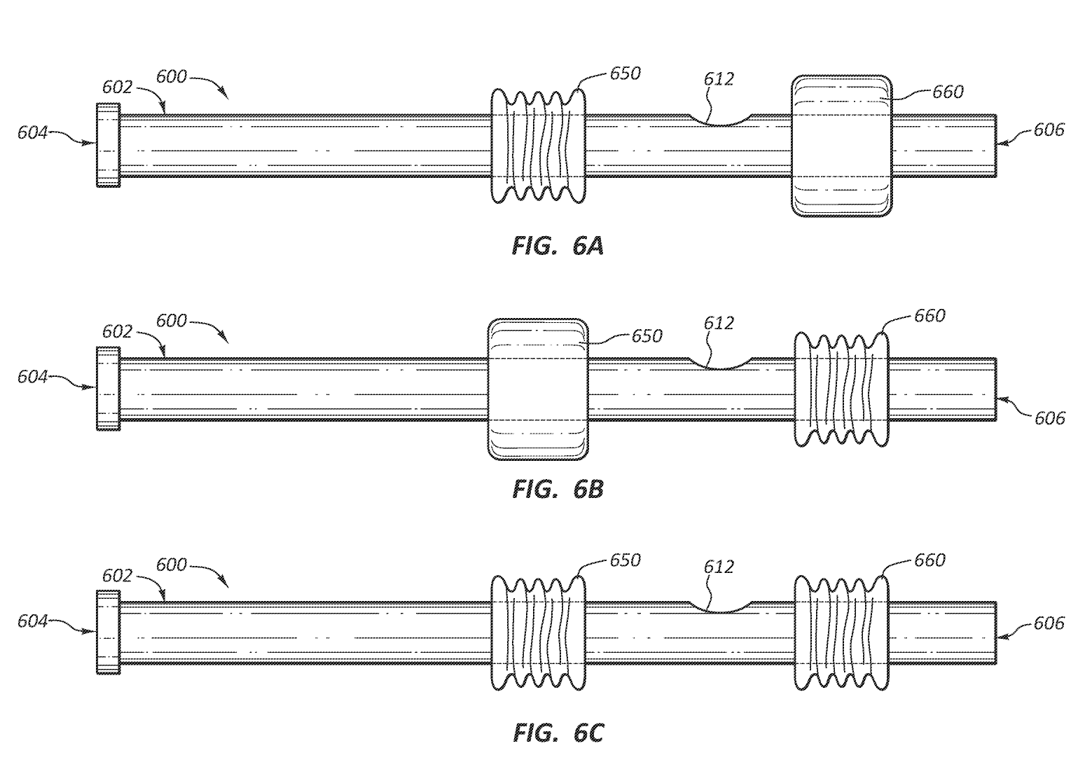

FIGS. 6A, 6B, and 6C are side views of a retrieval device having a distal opening 612 from which to deploy a medical device and two balloons: a proximal balloon 650, located proximally of the opening 612, and a distal balloon 660, located distally of the opening 612. Referring specifically to FIG. 6A, the proximal balloon 650 is partially inflated and the distal balloon 660 is fully inflated. During therapy, a practitioner may only partially inflate one or both balloons, as shown in FIGS. 6A-6C. In some instances, a practitioner may do so in order to partially stabilize the retrieval device while still allowing some fluid flow through the body lumen. Likewise, a practitioner may fully inflate one or both balloons, then partially deflate one or both balloons at one or more times during the procedure in order to, for example, allow some fluid to flow past the balloons and decrease pressure upstream of the balloons. Similarly, a retrieval device having only one balloon may be partially inflated or partially deflated during therapy.

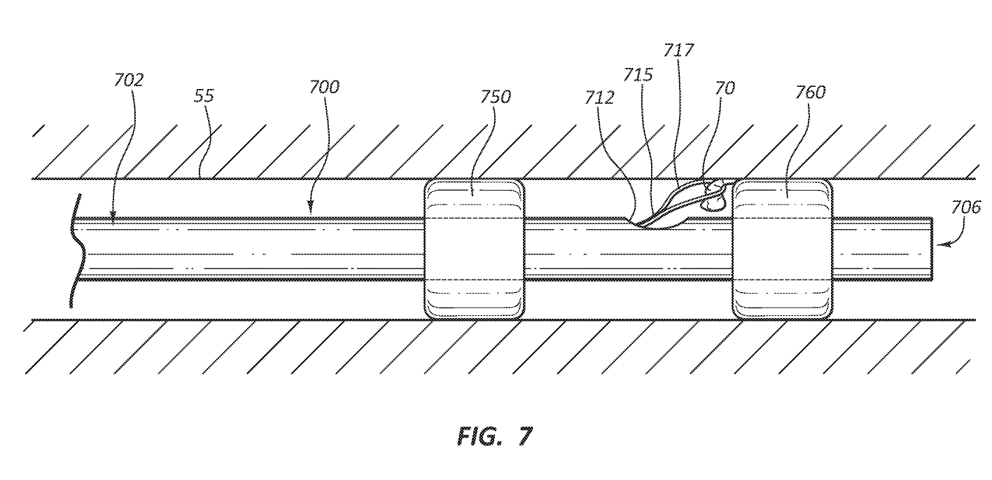

FIG. 7 is a side view of a retrieval device 700 disposed within a body lumen 50. The retrieval device 700 includes two balloons, a proximal balloon 750 and a distal balloon 760. As illustrated, both balloons 750, 760 are inflated such that each balloon is in contact with the sidewall 55 of the body lumen 50. As illustrated in FIG. 7, a snare device 715 is deployed from an opening 712 in a device lumen. The snare device 715 includes a snare loop 717, which is surrounding a fragment 70. The snare loop may be utilized to, for example, sever the fragment, sever and capture the fragment, or any other similar therapy.

Without further elaboration, it is believed that one skilled in the art can use the preceding description to utilize the present disclosure to its fullest extent. The examples and embodiments disclosed herein are to be construed as merely illustrative and exemplary, and not a limitation of the scope of the present disclosure in any way. It will be apparent to those having skill in the art that changes may be made to the details of the above-described embodiments without departing from the underlying principles of the disclosure herein. It is intended that the scope of the invention be defined by the claims appended hereto and their equivalents.

1. A snare assembly, comprising:
an elongate outer sheath having proximal and distal ends;
a first lumen disposed within the outer sheath between the proximal and distal ends of the outer sheath, the first lumen having a proximal opening adjacent the proximal end of the outer sheath and a distal opening adjacent the distal end of the outer sheath;
a snare having an elongate body portion and a first snare loop coupled to an end of the body portion, the snare disposed within the first lumen of the outer sheath;
a second lumen disposed within the outer sheath between the proximal and distal ends of the outer sheath; and
a first balloon coupled to the outer sheath adjacent the distal end of the outer sheath, the first balloon in fluid communication with the second lumen, and the first balloon configured to expand radially outward from the outer sheath.

2. The snare assembly of claim 1, wherein the first balloon is coupled to the outer sheath distal the distal opening of the first lumen.

3. The snare assembly of claim 1, wherein the first balloon is coupled to the outer sheath proximal the distal opening of the first lumen.

4. The snare assembly of claim 1, wherein the distal opening of the first lumen is located in a sidewall of the outer sheath.

5. The snare assembly of claim 1, further comprising a third lumen disposed within the outer sheath between the proximal and distal ends of the outer sheath.

6. The snare assembly of claim 5, wherein the first lumen has a diameter between about 0.025 inches and 0.110 inches.

7. The snare assembly of claim 5, wherein the second lumen has a diameter between about 0.002 inches and 0.009 inches.

8. The snare assembly of claim 5, wherein the third lumen has a diameter between about 0.012 inches and 0.044 inches.

9. The snare assembly of claim 5, further comprising a guidewire disposed within the third lumen.

10. The snare assembly of claim 5, further comprising a second balloon coupled to the outer sheath adjacent the distal end of the outer sheath, the second balloon in fluid communication with the third lumen, and the second balloon configured to expand radially outward from the outer sheath,
wherein the first balloon is coupled to the outer sheath proximal of the distal opening of the first lumen and the second balloon is coupled to the outer sheath distal the distal opening of the first lumen.

11. The snare assembly of claim 9, further comprising,
a fourth lumen disposed within the outer sheath between the proximal and distal ends of the outer sheath; and
a second balloon coupled to the outer sheath adjacent the distal end of the outer sheath, the second balloon in fluid communication with the fourth lumen, and the second balloon configured to expand radially outward from the outer sheath,
wherein the first balloon is coupled to the outer sheath proximal of the distal opening of the first lumen and the second balloon is coupled to the elongate outer sheath distal the distal opening of the first lumen.

12. A catheter assembly, comprising:
an elongate outer sheath having proximal and distal ends;
a first lumen disposed within the outer sheath between the proximal and distal ends of the outer sheath, the first lumen having a proximal opening adjacent the proximal end of the outer sheath and a distal opening adjacent the distal end of the outer sheath, the distal opening of the first lumen located in a sidewall of the outer sheath, and the first lumen configured to receive a surgical tool configured to cut or capture material or fragments within a body lumen;
a second lumen disposed within the outer sheath between the proximal and distal ends of the outer sheath;
a first balloon coupled to the outer sheath adjacent the distal end of the outer sheath, the first balloon in fluid communication with the second lumen, and the first balloon configured to expand radially outward from the outer sheath; and
a third lumen disposed within the elongate outer sheath between the proximal and distal ends of the elongate outer sheath, the third lumen configured to receive a guidewire.

13. The catheter assembly of claim 12, wherein the first balloon is coupled to the outer sheath distal the distal opening of the first lumen.

14. The catheter assembly of claim 12, wherein the first balloon is coupled to the outer sheath proximal the distal opening of the first lumen.

15. The catheter assembly of claim 12, further comprising,
a fourth lumen disposed within the outer sheath between the proximal and distal ends of the outer sheath, and
a second balloon coupled to the outer sheath adjacent the distal end of the outer sheath, the second balloon in fluid communication with the fourth lumen, and the second balloon configured to expand radially outward from the outer sheath,
wherein the first balloon is coupled to the outer sheath proximal of the distal opening of the first lumen and the second balloon is coupled to the outer sheath distal the distal opening of the first lumen.

16. A method of manipulating a snare within a body lumen of a patient, comprising:
placing an elongate catheter within a body lumen, the catheter having proximal and distal ends;
inflating a first balloon coupled to the catheter;
using a snare disposed within a first lumen of the elongate catheter to perform a therapeutic procedure, the first lumen having a distal opening adjacent the distal end of the elongate catheter.

17. The method of claim 16, wherein the first balloon is only partially inflated.

18. The method of claim 16, wherein the first balloon is at least partially inflated, then at least partially deflated, then at least partially re-inflated at least once during the course of the therapeutic procedure.

19. The method of claim 16, further comprising inflating a second balloon coupled to the catheter.

20. The method of claim 19, wherein the first balloon is coupled to the catheter at a location distal the distal opening of the first lumen and the second balloon is coupled to the catheter at a location proximal the distal opening of the first lumen.

21. The method of claim 19, wherein at least one of the first balloon and the second balloon is only partially inflated.

22. The method of claim 16, wherein a guidewire is disposed within a lumen of the catheter.
